(19) United States
(12) Patent Application Publication
(10) Pub. No.: US 2026/0315659 A1
(43) Pub. Date: Oct. 15, 2026

(54) MATERIALS FOR TISSUE ENGINEERING AND DISEASE THERAPEUTICS

(71) Applicants: The Regents of the University of Michigan, Ann Arbor, MI (US); The Board of Trustees of the University of Illinois, Urbana, IL (US)

(72) Inventors: Sung-Won HWANG, Ann Arbor, MI (US); Cong Truc HUYNH, Countryside, IL (US); Eben ALSBERG, Evanston, IL (US); Allen Po-Chih LIU, Ann Arbor, MI (US)

(73) Assignees: The Regents of the University of Michigan, Ann Arbor, MI (US); The Board of Trustees of the University of Illinois, Urbana, IL (US)

(21) Appl. No.: 19/485,350

(22) PCT Filed: May 17, 2024
(86) PCT No.: PCT/US2024/030002

§ 371 Date: Nov. 18, 2025

Related U.S. Application Data
(60) Provisional application No. 63/467,814, filed on May 19, 2023.

(51) Int. Cl. A61L 27/52; A61K 8/04; A61K 9/06; A61K 9/127; A61K 49/00; A61L 27/44; A61L 27/54; A61Q 19/00

(52) U.S. Cl. CPC A61L 27/52; A61K 8/042; A61K 9/06; A61K 9/127; A61K 49/0002; A61L 27/44; A61L 27/54; A61Q 19/00

(57) ABSTRACT

A material for therapeutics or tissue engineering includes a biocompatible hydrogel matrix and a plurality of vesicles distributed within the biocompatible hydrogel matrix. Each of the vesicles includes a shell and a core region. The shell includes a bilayer including a lipid and a block co-polymer. The shell is configured to release one or more compounds from the core region in response to an external stimulus. The core region includes the one or more compounds to be released into the biocompatible hydrogel matrix.

### CROSS-REFERENCE TO RELATED APPLICATIONS

This application claims the benefit of U.S. Provisional Application No. 63/467,814 filed on May 19, 2023. The entire disclosure of the above application is incorporated herein by reference.

### GOVERNMENT FUNDING

This invention was made with government support under AR080363 awarded by the National Institutes of Health. The government has certain rights in the invention.

### FIELD

The present disclosure relates to tissue engineering, and more particularly, to biomimetic, mechanoresponsive materials that may be used for partial or full reconstructing of a specific region of a human or animal subject, including, for example, bone, cartilage, or muscle.

### BACKGROUND

This section provides background information related to the present disclosure which is not necessarily prior art.

The number of patients waiting for organ transplantation far exceeds the number of available donated organs. Tissue engineering, which aims to develop functional substitute tissues via multiple-step processes using combination of cells, bioactive factors, and/or biocompatible scaffolds, could address the currently unmet medical need to restore functionality for tissues damaged or lost due to disease, injury, or congenital defects. Tissue engineering approaches often involve either engineering the needed tissue in vitro over the course of weeks or months followed by in vivo implantation, or more rapid implantation of a construct at the defect site and using signals from a scaffold including, for example, incorporated cells and/or soluble factors and signals emitted at the defect site to drive new tissue formation. Scaffold degradation can dynamically affect scaffold mechanical properties and has been demonstrated to regulate the behavior of host and transplanted cells, such as spreading, proliferation, migration, and differentiation. It is often difficult to control the degradation of employed biodegradable scaffolds after implantation. Accordingly, it would be desirable to have materials for tissue engineering that allow for regulation of degradation based on exogenous or external stimuli, such as enzymes, pH, light, and/or mechanical stress.

### SUMMARY

This section provides a general summary of the disclosure and is not a comprehensive disclosure of its full scope or all of its features.

In various aspects, the present disclosure provides a material for therapeutics or tissue engineering.

In one aspect, the material may include a biocompatible hydrogel matrix and a plurality of vesicles distributed within the biocompatible hydrogel matrix. Each of the vesicles may include a shell and a core region. The shell may include a bilayer including a lipid and a block co-polymer. The shell may be configured to release one or more compounds from the core region in response to an external stimulus. The core region includes the one or more compounds to be released into the biocompatible hydrogel matrix and surrounding environment.

In one aspect, the biocompatible hydrogel matrix may include (i) a polysaccharide selected from the group consisting of dextran, starch, cellulose, alginate, hyaluronic acid, chitosan, chitin, pectin, derivatives thereof, and combinations thereof; (ii) a polypeptide selected from the group consisting of collagen, fibronectin, gelatin, derivatives thereof, and combinations thereof; (iii) a synthetic polymer selected from the group consisting of: polyethylene glycol (PEG), poly(vinyl alcohol) (PVA), poly(2-hydroxyethyl methacrylate) (PHEMA), polyvinylpyrrolidone (PVP), polyimide (PI), polyamide (PA), polyacrylic acid (PAA), polyurethane (PU), polyesters (such as, polycaprolactone (PCL), polylactic acid (PLA), poly(caprolactone-co-lactic acid) (PCLA), and/or poly(lactic-co-glycolic-acid) (PLGA)), derivatives thereof, and combinations thereof; or (iv) any combination of (i)-(iii).

In one aspect, the biocompatible hydrogel matrix may include less than 40 wt. % of a polymer selected from the group consisting of: dextran, starch, cellulose, alginate, hyaluronic acid, chitosan, chitin, pectin, collagen, fibronectin, gelatin, polyethylene glycol (PEG), poly(vinyl alcohol) (PVA), poly(2-hydroxyethyl methacrylate) (PHEMA), polyvinylpyrrolidone (PVP), polyimide (PI), polyamide (PA), polyacrylic acid (PAA), polyurethane (PU), polyesters (such as, polycaprolactone (PCL), polylactic acid (PLA), poly(caprolactone-co-lactic acid) (PCLA), and/or poly(lactic-co-glycolic-acid) (PLGA)), derivatives thereof, and combinations thereof.

In one aspect, the one or more compounds may include a bioactive agent selected from the group consisting of: a cell adhesion factor, a growth factor, a peptide, a cytokine, a hormone, a pharmaceutical agent, and combinations thereof.

In one aspect, the one or more compounds may include a bioactive agent selected from the group consisting of: a cell adhesion factor, a growth factor, a peptide, a cytokine, a hormone, a pharmaceutical agent, matrix metalloproteinases (MMPs), plasmid DNA (pDNA), messenger ribonucleic acid (mRNA), strong interfering RNA (siRNA), micro RNA (miRNA), biochemicals, oligonucleotides, and combinations thereof.

In one aspect, the one or more compounds may include an active ingredient selected from the group consisting of: a therapeutic active ingredient, a systemic active ingredient, a chemotherapy active ingredient, a localized active ingredient, an oral care active ingredient, a nutritional active ingredient, a personal care active ingredient, a cosmetic active ingredient, a diagnostic imaging indicator agent, and combinations thereof.

In one aspect, the lipid may be selected from the group consisting of: dipalmitoylphosphatidylcholine (DPPC), 1,2-dioleoyl-sn-glycero-3-phosphocholine (DOPC), distearoylphosphatidylcholine (DSPC), phosphatidylcholine (POPC), 1,2-distearoyl-sn-glyero-3-phosphoethanolamine (DSPE), 1,2-dilauroyl-sn-glycero-3-phosphocholine (DLPC), cholesterol, egg phosphatidylcholine (Egg PC), derivatives thereof, and combinations thereof.

In one aspect, the block co-polymer may be selected from the group consisting of: polybutadiene (PBD)-b-PEG, polyisobutylene (PIB)-b-PEG, triazine-functionalized (TRI)-PEG-b-PIB, oligo(alkaline surfactant polymer (Asp))-b-poly(phenylene oxide) (PPhO), monomethyl ethyl PEG (mPEG)-b-poly((methyl methacrylate (MMA)-grad-(2-(dimethylamino)ethyl methacrylate (DMAEMA)) (P(MMA-grad-DMAEMA)), PEG-poly(propylene glycol) (PPO)-PEG, PPO-PEG-PPO, mPEG-b-PCL, PEG-b-PCL, PEG-b-PCL-b-PEG, PEG-b-PLA, polydimethylsiloxane (PDMS)-b-PEG, poly(2-methyl-2-oxazoline) (PMOXA)-b-PDMS-b-PMOXA, PEG-b-PPhO-b-PEG, poly(glycerol monomethacrylate) (PGM)-b-PPhO-b-PGM, PEG-b-PDMS-b-PEG, PDMS-g-(PEG)₂₋₃, PEG-g-PDMS-g-PEG, derivatives thereof, and combinations thereof.

In one aspect, the block co-polymer may be selected from the group consisting of: PBD-b-PEO, PIB-b-PEO, TRI-PEO-b-PIB, oligo(Asp)-b-PPO, mPEG-b-P(MMA-grad-DMAEMA), mPEG-b-PCL, PEG-b-PCL, PEG-b-PLA, PEG-b-PBD, PDMS-b-PEO, PMOXA-b-PDMS-b-PMOXA, PEO-b-PPO-b-PEO, PGM-b-PPO-b-PGM, PEO-b-PDMS-b-PEO, PEO-b-PCL-b-PEO, PDMS-g-(PEO)₂, PDMS-g-(PEO)₂₋₃, PEO-b-PCL, PEG-b-PDMS-b-PEG, PEG-g-PDMS-g-PEG, derivatives thereof, and combinations thereof.

In one aspect, a mole fraction of the lipid to the block co-polymer in the shell may be greater than or equal to about 1:99 to less than or equal to about 99:1.

In one aspect, a mole fraction of the lipid to the block co-polymer in the shell may be greater than or equal to about 10:90 to less than or equal to about 90:10.

In one aspect, the external stimulus may increase a porosity of the shell to release the one or more compounds from the core region.

In one aspect, the external stimulus may rupture the shell to release the one or more compound from the core region.

In one aspect, the external stimulus may be selected from the group consisting of: applied energy fields, electrical fields, magnetic fields, pH, humidity, stress, light, temperature, sound, cell secreted factors, enzymes, biomolecules, and combinations thereof.

In one aspect, the external stimulus may include a stress that is applied at greater than or equal to about 1 Pa to less than or equal to about 1,000 MPa.

In one aspect, the shell may further include a permanent super magnetic material and the external stimulus comprises a magnetic field.

In one aspect, an average diameter of the plurality of vesicles may be greater than 1 micrometer to less than or equal to about 1 millimeter.

In one aspect, an average diameter of the plurality of vesicles may be greater than 1 micrometer to less than or equal to about 100 micrometers.

In one aspect, the material further includes one or more cells distributed in the biocompatible hydrogel matrix.

In various aspects, the present disclosure provides a method for preparing a material for tissue engineering.

In one aspect, the method may include preparing a plurality of vesicles and disposing the plurality of vesicles in a biocompatible hydrogel matrix. Each vesicle may include a shell and a core region. The shell may include a bilayer that includes a lipid and a block co-polymer. The core region may include one or more compounds. The preparing may include adding a first solution and a second solution to a chamber to form an oil-water interface, where the second solution includes the lipid and the block co-polymer; adding water-in-oil monolayer emulsion droplets include the second solution and a third solution to the chamber, where the third solution includes the one or more compounds, and the first solution has an osmolality that is greater than or equal to about 1% to less than or equal to about 50% greater than the third solution; and applying a centrifugal force to the chamber such that lipid and the block co-polymer for the bilayer.

In one aspect, the third solution may have an osmolality greater than or equal to about 200 mOsm/kg to less than or equal to about 1,000 mOsm/kg.

In one aspect, the first solution may have an osmolality greater than or equal to about 202 mOsm/kg to less than or equal to about 1,500 mOsm/kg.

In one aspect, the second solution may include greater than or equal to about 1 mol. % to less than or equal to about 99 mol. % of the lipid and greater than or equal to about 1 mol. % to less than or equal to about 99 mol. % of the block co-polymer.

In one aspect, the water-in-oil monolayer emulsion droplets may include greater than or equal to about 80 vol. % to less than or equal to about 99 vol. % of the second solution and greater than or equal to about 1 vol. % to less than or equal to about 20 vol. % of the third solution.

In one aspect, the lipid may be selected from the group consisting of: dipalmitoylphosphatidylcholine (DPPC), 1,2-dioleoyl-sn-glycero-3-phosphocholine (DOPC), distearoylphosphatidylcholine (DSPC), phosphatidylcholine (POPC), 1,2-distearoyl-sn-glyero-3-phosphoethanolamine (DSPE), 1,2-dilauroyl-sn-glycero-3-phosphocholine (DLPC), cholesterol, egg phosphatidylcholine (Egg PC), derivatives thereof, and combinations thereof.

In one aspect, the block co-polymer may be selected from the group consisting of: polybutadiene (PBD)-b-PEG, polyisobutylene (PIB)-b-PEG, triazine-functionalized (TRI)-PEG-b-PIB, oligo(alkaline surfactant polymer (Asp))-b-poly(phenylene oxide) (PPhO), monomethyl ethyl PEG (mPEG)-b-poly((methyl methacrylate (MMA)-grad-(2-(dimethylamino)ethyl methacrylate (DMAEMA)) (P(MMA-grad-DMAEMA)), PEG-poly(propylene glycol) (PPO)-PEG, PPO-PEG-PPO, mPEG-b-PCL, PEG-b-PCL, PEG-b-PCL-b-PEG, PEG-b-PLA, polydimethylsiloxane (PDMS)-b-PEG, poly(2-methyl-2-oxazoline) (PMOXA)-b-PDMS-b-PMOXA, PEG-b-PPhO-b-PEG, poly(glycerol monomethacrylate) (PGM)-b-PPhO-b-PGM, PEG-b-PDMS-b-PEG, PDMS-g-(PEG)₂₋₃, PEG-g-PDMS-g-PEG, derivatives thereof, and combinations thereof.

In one aspect, the block co-polymer may be selected from the group consisting of: PBD-b-PEO, PIB-b-PEO, TRI-PEO-b-PIB, oligo(Asp)-b-PPO, mPEG-b-P(MMA-grad-DMAEMA), mPEG-b-PCL, PEG-b-PCL, PEG-b-PLA, PEG-b-PBD, PDMS-b-PEO, PMOXA-b-PDMS-b-PMOXA, PEO-b-PPO-b-PEO, PGM-b-PPO-b-PGM, PEO-b-PDMS-b-PEO, PEO-b-PCL-b-PEO, PDMS-g-(PEO)₂, PDMS-g-(PEO)₂₋₃, PEO-b-PCL, PEG-b-PDMS-b-PEG, PEG-g-PDMS-g-PEG, derivatives thereof, and combinations thereof.

In one aspect, the one or more compounds may include a bioactive agent selected from the group consisting of: a cell adhesion factor, a growth factor, a peptide, a cytokine, a hormone, a pharmaceutical agent, and combinations thereof.

In one aspect, the one or more compounds may include a bioactive agent selected from the group consisting of: a cell adhesion factor, a growth factor, a peptide, a cytokine, a hormone, a pharmaceutical agent, matrix metalloproteinases (MMPs), plasmid DNA (pDNA), messenger ribonucleic acid (mRNA), strong interfering RNA (siRNA), micro RNA (miRNA), biochemicals, oligonucleotides, and combinations thereof.

In one aspect, the one or more compounds may include a therapeutic active ingredient, a systemic active ingredient, a chemotherapy active ingredient, a localized active ingredient, an oral care active ingredient, a nutritional active ingredient, a personal care active ingredient, a cosmetic active ingredient, a diagnostic imaging indicator agent, and combinations thereof.

In one aspect, the biocompatible hydrogel matrix may include less than 40 wt. % of a polymer selected from the group consisting of: dextran, starch, cellulose, alginate, hyaluronic acid, chitosan, chitin, pectin, collagen, fibronectin, gelatin, polyethylene glycol (PEG), poly(vinyl alcohol) (PVA), poly(2-hydroxyethyl methacrylate) (PHEMA), polyvinylpyrrolidone (PVP), polyimide (PI), polyamide (PA), polyacrylic acid (PAA), polyurethane (PU), polyesters (such as, polycaprolactone (PCL), polylactic acid (PLA), poly(caprolactone-co-lactic acid) (PCLA), and/or poly(lactic-co-glycolic-acid) (PLGA)), derivatives thereof, and combinations thereof.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1A is a simple illustration of an example material in a closed or pre-treatment state for tissue engineering and for partial or full reconstructing of a specific region of a human or animal subject in accordance with various aspects of the present disclosure;

FIG. 1B is an illustration of the example material of FIG. 1A in an open or treatment state in accordance with various aspects of the present disclosure; and

FIG. 2 is a flowchart illustrating an example process for preparing an example material for tissue engineering, for partial or full reconstructing of a specific region of a human or animal subject in accordance with various aspects of the present disclosure.

Corresponding reference numerals indicate corresponding parts throughout the several views of the drawings.

### DETAILED DESCRIPTION

Example embodiments are provided so that this disclosure will be thorough and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected, or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer, or section. Terms such as “first,” “second,” and other numerical terms when used herein do not imply a sequence or order unless clearly indicated by the context. Thus, a first step, element, component, region, layer, or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

The present disclosure contemplates a material for tissue engineering, and more specifically, to biomimetic, mechanoresponsive materials that may include hydrogels, for partial or full reconstructing of a specific region of a human or animal subject, including, for example, bone, cartilage, or muscle, that are capable of adapting their physical and chemical properties in response to an exogenous or external stimulus, including, for example, external mechanical stimuli. Such biomimetic, mechanoresponsive materials have important biomedical applications in wound repair, drug delivery, tissue scaffold, and artificial tissue.

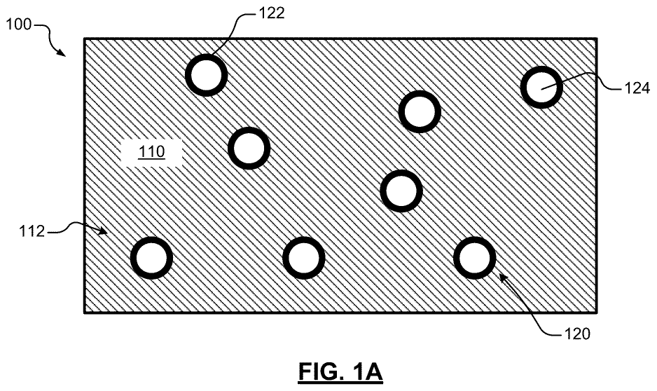

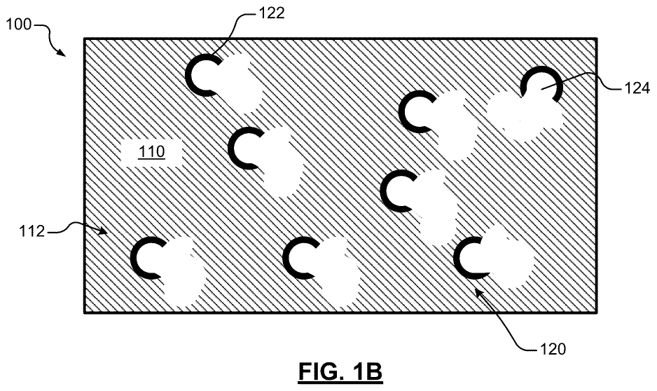

In various aspects, as illustrated in FIG. 1A, a material 100 for tissue engineering includes a hydrogel 112 and a plurality of vesicles (also referred to as synthetic cells) 120 distributed within the hydrogel 112. In certain variations, the vesicles 120 may occupy greater than 0 volume percent (vol. %) to less than or equal to about 30 vol. %, optionally greater than 0 vol. % to less than or equal to about 20 vol. %, and in certain aspects optionally greater than or equal to about 5 vol. % to less than or equal to about 20 vol. %, of a total volume of the material 100. The inclusion of the plurality of vesicles 120 does not require chemical modification of the hydrogel 112.

The hydrogel 112 may include a porous three-dimensional matrix (e.g., casted into using a solvent) a porous three-dimensional matrix 110. The plurality of vesicles 120 may be distributed within the hydrogel matrix 110. Although not illustrated, in certain variations, the hydrogel 112 may also include a one or more cells distributed therewithin. In certain aspects, the plurality of vesicles 120 (and optionally the one or more cells) may be distributed evenly within the hydrogel 112, for example, homogenously distributed therein. In other variations, the vesicles 120 (and optionally the one or more cells) may be unevenly or randomly distributed, or alternatively, distributed in a predetermined concentration gradient, for example, having a greater concentration in one region or portion that transitions to a reduced concentration in a distinct region or portion of the material 100. In still other variations, the vesicles 120 (and optionally, the one or more cells) may be distributed to form a desired pattern.

Hydrogel matrices, like the hydrogel matrix 110, are formed from lightly crosslinked networks of natural and/or synthetic polymers that may have high water contents. For example, in certain variations, the hydrogel matrices may have a water content that is about 60 weight percent (wt. %) per volume or more, optionally about 70 wt. % per volume or more, optionally about 80 wt. % per volume or more, and in certain aspects, optionally 90 wt. %, per volume or more. The polymeric crosslinking defining the hydrogel matrices can be achieved by various methods including hydrophobic or ionic interactions, covalent chemical, or ultraviolet (UV)-initiated chemical crosslinking. Additionally, or alternatively, the polymeric crosslinking defining the hydrogel matrices can be achieved by thermogelling and/or IPN. The method and degree of crosslinking can be varied to affect that degradation or permanence of the hydrogel.

The natural polymers, as included in the hydrogel 112, may include, for example, polysaccharides and/or polypeptides. The polysaccharides may be selected from the group consisting of dextran, starch, cellulose, alginate, hyaluronic acid, chitosan, chitin, pectin, derivatives thereof, and combinations thereof. The polypeptides may be selected from the group consisting of: collagen, fibronectin, gelatin, derivatives thereof, and combinations thereof. The synthetic polymers as included in the hydrogel 112 may include, for example, polyethylene glycol (PEG), poly(vinyl alcohol) (PVA), poly(2-hydroxyethyl methacrylate) (PHEMA), polyvinylpyrrolidone (PVP), polyimide (PI), polyamide (PA), polyacrylic acid (PAA), polyurethane (PU), polyesters (such as, polycaprolactone (PCL), polylactic acid (PLA), poly(caprolactone-co-lactic acid) (PCLA), and/or poly(lactic-co-glycolic-acid) (PLGA)), derivatives thereof, and combinations thereof.

In certain variations, the natural polymers may be selected from the group consisting of: dextran, starch, cellulose, alginate, hyaluronic acid, chitosan, chitin, pectin, collagen, fibronectin, gelatin, derivatives thereof, and combinations thereof. In certain variations, the synthetic polymers may be selected from the group consisting of: polyethylene glycol (PEG), poly(vinyl alcohol) (PVA), poly(2-hydroxyethyl methacrylate) (PHEMA), polyvinylpyrrolidone (PVP), polyimide (PI), polyamide (PA), polyacrylic acid (PAA), polyurethane (PU), polyesters, derivatives thereof, and combinations thereof. In certain variations, the synthetic polymers may be selected from the group consisting of: polyethylene glycol (PEG), poly(vinyl alcohol) (PVA), poly(2-hydroxyethyl methacrylate) (PHEMA), polyvinylpyrrolidone (PVP), polyimide (PI), polyamide (PA), polyacrylic acid (PAA), polyurethane (PU), polycaprolactone (PCL), polylactic acid (PLA), poly(caprolactone-co-lactic acid) (PCLA), poly(lactic-co-glycolic-acid (PLGA), derivatives thereof, and combinations thereof.

In certain variations, the hydrogel 112 may include a biocompatible polymer selected from the group consisting of: dextran, starch, cellulose, alginate, hyaluronic acid, chitosan, chitin, pectin, collagen, fibronectin, gelatin, polyethylene glycol (PEG), poly(vinyl alcohol) (PVA), poly(2-hydroxyethyl methacrylate) (PHEMA), polyvinylpyrrolidone (PVP), polyimide (PI), polyamide (PA), polyacrylic acid (PAA), polyurethane (PU), polyesters, derivatives thereof, and combinations thereof. In certain variations, the hydrogel 112 may include a biocompatible polymer selected from the group consisting of: polyethylene glycol (PEG), poly(vinyl alcohol) (PVA), poly(2-hydroxyethyl methacrylate) (PHEMA), polyvinylpyrrolidone (PVP), polyimide (PI), polyamide (PA), polyacrylic acid (PAA), polyurethane (PU), polycaprolactone (PCL), polylactic acid (PLA), poly(caprolactone-co-lactic acid) (PCLA), poly(lactic-co-glycolic-acid) (PLGA), derivatives thereof, and combinations thereof. Biocompatible as used herein refers to non-toxic to cells and tissue of living organisms of the human or animal subject.

With renewed reference to FIG. 1A, as illustrated, each of the vesicles 120 of the plurality of vesicles may include a shell 122 and a core region 124. In certain aspects, the vesicles 120 are configured to release one or more compounds from the core region into the surrounding environment, for example, into the hydrogel 112. Thus, the core region 124 includes one or more compounds, which may be selected for delivery external to each vesicle and considered to be a “payload.”

The one or more compounds in the core region 124 may be selected to effect encapsulation or surrounding cell behavior, treat local or systemic disease, degrade the hydrogel 112 or otherwise modify or alter the properties of the hydrogel, and the like. In certain variations, for example, the one or more compounds in the core region 124 of the vesicles 120 may include an active ingredient. An active ingredient may be a compound or composition that diagnoses, prevents, or treats a physiological or psychological disorder or condition in, or can provide a cosmetic or aesthetic benefit to, the human or animal subject. The active ingredient may be targeted to a particular target, such as organs, tissues, medical implants or devices, hair, skin, mouth, eyes, circulatory system, and the like.

In certain variations, the one or more compounds in the core region 124 of the vesicles 120 may include an active ingredient selected from the group consisting of: a therapeutic active ingredient, a systemic active ingredient, a chemotherapy active ingredient, a localized active ingredient, an oral care active ingredient, a nutritional active ingredient, a personal care active ingredient, a cosmetic active ingredient, a diagnostic imaging indicator agent, and combinations thereof. In other variations, the one or more compounds may include, for example, a bioactive agent selected from the group consisting of: a cell adhesion factor, a growth factor, a peptide, a cytokine, a hormone, a pharmaceutical agent, and combinations thereof. The bioactive agent may additionally, or alternatively, include matrix metalloproteinases (MMPs), plasmid DNA (pDNA), messenger ribonucleic acid (mRNA), strong interfering RNA (siRNA), micro RNA (miRNA), biochemicals, oligonucleotides, and/or combinations thereof. Bioactive agents are those that have an effect on a living organism, tissue, and/or cell.

The shell 122 may include a lipid bilayer and a block co-polymer. For example, in certain variations, a mole fraction of the lipid bilayer to the block co-polymer in the shell is greater than or equal to about 1:99 to less than or equal to about 99:1, and in certain aspects, optionally greater than or equal to about 10:90 to less than or equal to about 90:10.

Lipid bilayers are thin hydrophobic, polar membranes including two layers of lipid molecules, where the two layers have an opposing structure. The lipid molecules are amphiphilic, including hydrophobic tails and hydrophilic heads attached thereto. The hydrophobic tails of the lipid molecules form a center of the lipid bilayers, and the hydrophilic heads form an exterior-facing surface of the lipid bilayer. Lipid bilayers are often used as a barrier to retain materials (including, for example, ions, proteins, and/or other molecules) within a desired region. The lipid bilayers include phospholipids such as dipalmitoylphosphatidylcholine (DPPC), 1,2-dioleoyl-sn-glycero-3-phosphocholine (DOPC), distearoylphosphatidylcholine (DSPC), phosphatidylcholine (POPC), 1,2-distearoyl-sn-glyero-3-phosphoethanolamine (DSPE), 1,2-dilauroyl-sn-glycero-3-phosphocholine (DLPC), cholesterol, and/or egg phosphatidylcholine (Egg PC). In certain variations, the shell 122 may include lipids selected from the group consisting of: dipalmitoylphosphatidylcholine (DPPC), 1,2-dioleoyl-sn-glycero-3-phosphocholine (DOPC), distearoylphosphatidylcholine (DSPC), phosphatidylcholine (POPC), 1,2-distearoyl-sn-glyero-3-phosphoethanolamine (DSPE), 1,2-dilauroyl-sn-glycero-3-phosphocholine (DLPC), cholesterol, egg phosphatidylcholine (Egg PC), and combinations thereof.

Block co-polymers are macromolecules having a linear and/or radial arrangement of two or more different polymeric blocks joined by covalent bonds to form a single macromolecule. Like the lipid molecules, the macromolecules are amphiphilic, including hydrophobic blocks (such PBD) and hydrophilic blocks (such as PEG). The hydrophobic blocks define a center of the macromolecule, and the hydrophilic blocks face an exterior environment. The block co-polymers are mixed with phospholipids to generate hybrid bilayer membranes, where the block c-polymer helps to improve physicochemical stability, residence time in biological fluids, and reducing payload leakiness of the vesicles. Example block co-polymers include, for example, polybutadiene rubber (PBD)-b-PEO, polyisobutylene (PIB)-b-PEO, triazine-functionalized (TRI)-PEO-b-PIB, oligo(alkaline surfactant polymer (Asp))-b-polyphenylene oxide (PPO), monomethyl ethyl polyethylene glycol (mPEG)-b-poly(methyl methacrylate (MMA)-grad-(2-(dimethylamino)ethyl methacrylate (DMAEMA)), P(MMA-grad-DMAEMA), mPEG-b-PCL, PEG-b-PCL, PEG-b-PLA, PEG-b-PBD, polydimethylsiloxane (PDMS)-b-PEO, poly(2-methyl-2-oxazoline) (PMOXA)-b-PDMS-b-PMOXA, PEO-b-PPO-b-PEO, poly(glycerol monomethacrylate) (PGM)-b-PPO-b-PGM, PEO-b-PDMS-b-PEO, PEO-b-PCL-b-PEO, PDMS-g-(PEO)₂, PDMS-g-(PEO)₂₋₃, PEO-b-PCL, PEG-b-PDMS-b-PEG, and/or PEG-g-PDMS-g-PEG. In certain variations, the shell 122 may include block co-polymers selected from the group consisting of: polybutadiene (PBD)-b-PEG, polyisobutylene (PIB)-b-PEG, triazine-functionalized (TRI)-PEG-b-PIB, oligo(alkaline surfactant polymer (Asp))-b-poly(phenylene oxide) (PPhO), monomethyl ethyl PEG (mPEG)-b-poly((methyl methacrylate (MMA)-grad-(2-(dimethylamino)ethyl methacrylate (DMAEMA)) (P(MMA-grad-DMAEMA)), PEG-poly(propylene glycol) (PPO)-PEG, PPO-PEG-PPO, mPEG-b-PCL, PEG-b-PCL, PEG-b-PCL-b-PEG, PEG-b-PLA, polydimethylsiloxane (PDMS)-b-PEG, poly(2-methyl-2-oxazoline) (PMOXA)-b-PDMS-b-PMOXA, PEG-b-PPhO-b-PEG, poly(glycerol monomethacrylate) (PGM)-b-PPhO-b-PGM, PEG-b-PDMS-b-PEG, PDMS-g-(PEG)₂₋₃, PEG-g-PDMS-g-PEG, derivatives thereof, and combinations thereof. In certain variations, the shell 122 may include block co-polymers selected from the group consisting of: PBD-b-PEO, PIB-b-PEO, TRI-PEO-b-PIB, oligo(Asp)-b-PPO, mPEG-b-P(MMA-grad-DMAEMA), mPEG-b-PCL, PEG-b-PCL, PEG-b-PLA, PEG-b-PBD, PDMS-b-PEO, PMOXA-b-PDMS-b-PMOXA, PEO-b-PPO-b-PEO, PGM-b-PPO-b-PGM, PEO-b-PDMS-b-PEO, PEO-b-PCL-b-PEO, PDMS-g-(PEO)₂, PDMS-g-(PEO)₂₋₃, PEO-b-PCL, PEG-b-PDMS-b-PEG, PEG-g-PDMS-g-PEG, and combinations thereof.

An average particle diameter of the vesicles 120 may be greater than or equal to about 1 micrometer to less than or equal to about 1 millimeters, optionally greater than or equal to about 1 micrometer to less than or equal to about 100 micrometers, optionally greater than or equal to about 1 micrometer to less than or equal to about 50 micrometers, and in certain aspects, optionally greater than or equal to about 5 micrometers to less than or equal to about 30 micrometers. The shell 122 may have an average thickness greater than or equal to about 1 nanometer to less than or equal to about 20 nanometers, optionally greater than or equal to about 5 nanometers to less than or equal to about 10 nanometers, and in certain aspects, optionally about 5 nanometers. The selected block co-polymers may affect the thickness of the shell 122. The thickness of the shell 122 may depend on polymer molecular weight or degree of polymerization, particularly of the hydrophobic block of the block co-polymer.

The shell 122 is configured to release one or more compounds from the core region in response to being exposed to an exogenous or external stimulus. For example, collagen as included in the shell 122 may degrade in the presence of collagenases, gelatin as included in the shell 122 may degrade in response to temperature, nucleic acid sequences as included in the shell 122 may create porosity in response to base pair sequence, cell membrane shear stress responsive element as included in the shell 122 may respond to shear stress, bubbles as included in the shell 122 may burst in response to ultrasound, pH responsive polymers as included in the shell 122 may respond to a pH change, and the like.

In certain variations, as illustrated in FIG. 1B, the external stimulus may cause the shell 122 to undergo a mechanical change, such as a fissure, crack, rupture, or the like, releasing the one or more compounds from the core region 124. Although FIG. 1B illustrates a mechanical change, such as a fissure, crack, rupture, or the like, that releases the one or more compounds from the core region 124, it should be appreciated, that in other variations, the external stimulus may increase a porosity of the shell 122 to cause the one or more compounds to be released (e.g., by passing through the modified shell 122) from the core region 124. For example, the shell 122 may have an original or first porosity and, as the external stimulus is received, the shell 122 may have a second porosity that is greater than the first porosity. In each instance, the nature of the shell 122 may be tuned, for example, by modifying the ratio of the lipids to the block co-polymers to increase or decrease the rigidity of the shell 122. For example, a shell having a higher amount of the block co-polymer may have a higher rigidity. The size of the embedded vesicles 120 can also affect the degree of deformation of the shell 122. For example, larger vesicles may accept more pressure before deformation. Further still, the stiffness and the mechanical properties of the hydrogel 112 can also be adjusted (for example, based on hydrogel concentration and/or crosslinking density).

In various aspects, the external stimulus may be selected from the group consisting of: applied energy fields, electrical fields, magnetic fields, pH, humidity, stress (e.g., mechanical stress, compressive stress), light, temperature, sound, cell secreted factors, enzymes, biomolecules (e.g., proteins, genetic material), and combinations thereof. For example, shells including pH responsive polymers may go into solution in response to pH changes, while. Additionally, or alternatively, in certain variations, the external stimulus may include a stress (for example, a compression stress) that is applied at greater than or equal to about 1 pascal (Pa) to less than or equal to about 1,000 megapascal (MPa), optionally greater than or equal to about 1 kilopascal (kPa) to less than or equal to about 100 kPa, and in certain aspects, optionally greater than or equal to about 5 kPa to less than or equal to about 200 kPa. In certain variations, the stress may be applied by directly adding weight on top of the hydrogel.

In certain variations, the shell 122 may include one or more materials that are responsive to an external stimulus or force field. By way of example, the shell 122 may include a permanent super magnetic material and the external stimulus may include a magnetic field applied to change the porosity of the shell 122 and/or rupture the shell 122 when the material 100 is stimulated by a magnetic field (for example, using a permanent super paramagnetic bar and/or by placing the hydrogel on top of a table electrical stirrer). The permanent super magnetic material may include, for example, ferrites, nanoparticles, metallic nanoparticles, or derivatives thereof. The shell 122 may include greater than or equal to about 0.001 wt. % to less than or equal to about 0.1 wt. %, and in certain aspects, optionally greater than or equal to about 0.005 wt. % to less than or equal to about 0.05 wt. %, of the permanent super magnetic material.

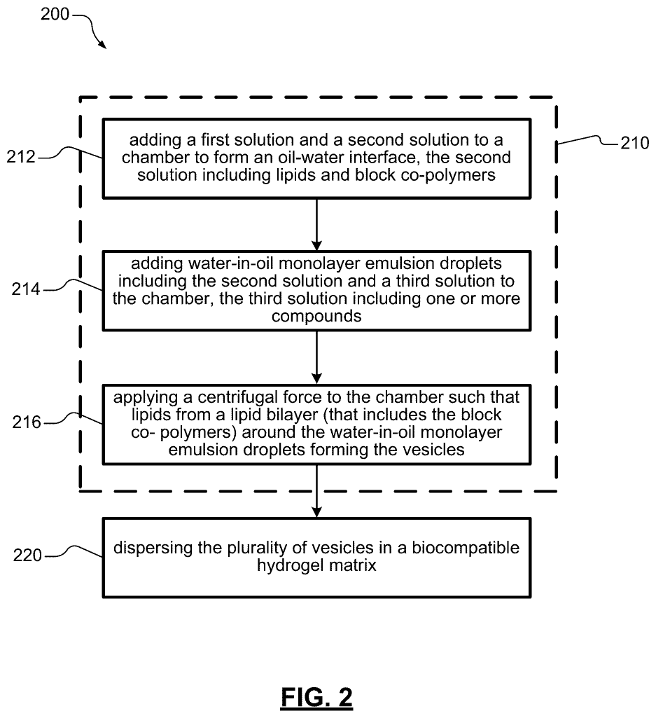

In various aspects, the present disclosure provides methods for preparing a material for tissue engineering, and more specifically, to biomimetic, mechanoresponsive hydrogels, for partial or full reconstructing of a specific region of a human or animal subject, like the material 100 illustrated in FIG. 1A. An example method 200 is illustrated in FIG. 2. The method 200 may include preparing 210 a plurality of vesicles, each vesicle of the plurality including a shell and a core region, like the vesicles 120 illustrated in FIG. 1A, and dispersing (e.g., encapsulating) 220 the plurality of vesicles in a biocompatible hydrogel matrix, like the hydrogel matrix 112 illustrated in FIG. 1A.

In certain variations, the preparing 210 of the vesicles may include contacting 212 a first solution and a second solution within a chamber to form an oil-water interface. The second solution, which may also be referred to as a lipid-in-oil dispersion, may include lipids and block co-polymers, like those detailed above. Although not illustrated, it should be appreciated that in certain variations, the method 200 may include preparing the second solution. The preparing of the second solution may include contacting lipids and block co-polymers. In certain variations, contacting the lipids and block co-polymers may including adding the lipids and block co-polymers, consecutively or consequentially to a stock solution and then drying the solution, for example, using a gentle argon flow. In certain variations, the block co-polymers may be provided as part of the stock solution and contacting the lipids and block co-polymers may including adding the lipids to the stock solution and/or adding the lipids and the stock solution to a container consecutively or concurrently and then drying the solution, for example, using a gentle argon flow. In each instance, the lipids and block co-polymers may be re-dissolved in organic solvent (including, for example, chloroform) and dispersed in an oil mix (including, for example, a volumetric ratio of silicone oil and mineral oil of about 4:1). The second solution may include greater than or equal to about 1 mole percent (mol. %) to less than or equal to about 99 mol. %, and in certain aspects, optionally greater than or equal to about 10 mol. % to less than or equal to about 90 mol. %, of the lipids; and greater than or equal to about 1 mol. % to less than or equal to about 99 mol. %, and in certain aspects, optionally greater than or equal to about 10 mol. % to less than or equal to about 90 mol. %, of the block co-polymers.

With renewed reference to FIG. 2, the preparing 210 of the vesicle may include adding water-in-oil monolayer emulsion droplets that include the second solution and a third solution into the chamber. The water-in-oil monolayer emulsion droplets may be added to the chamber using a dropwise process. Although not illustrated, it should be appreciated that in certain variations, the method 200 may include preparing the water-in-oil monolayer emulsion droplets. The preparing of the water-in-oil monolayer emulsion droplets may include contacting the first and third solutions and pipetting the mixture up and down to produce the water-in-oil monolayer emulsion droplets. The water-in-oil monolayer emulsion droplets may include greater than or equal to about 80 vol. % to less than or equal to about 99 vol. %, and in certain aspects, optionally include greater than or equal to about 82 vol. % to less than or equal to about 97 vol. %, of the second solution; and greater than or equal to about 1 vol. % to less than or equal to about 20 vol. %, and in certain aspects, optionally include greater than or equal to about 3 vol. % to less than or equal to about 18 vol. %, of the third solution.

The third solution, which may also be referred to an inner encapsulation solution, may include the one or more compounds. The third solution may have an osmolality greater than or equal to about 200 milliosmole per kilogram (mOsm/kg) to less than or equal to about 1,000 mOsm/kg. The first solution may have an osmolality that is greater than or equal to about 1% to less than or equal to about 50%, optionally greater than or equal to about 1% to less than or equal to about 20%, and in certain aspects, optionally, greater than or equal to about 5% to less than or equal to about 10% greater than the third solution. For example, the first solution may have osmolality greater than or equal to about 202 mOsm/kg to less than or equal to about 1,500 mOsm/kg, and in certain aspects, optionally greater than or equal to about 210 mOsm/kg to less than or equal to about 1,100 mOsm/kg. The first solution, which may also be referred to as an outer solution, includes a diluted buffer stock solution. Although not illustrated, it should be appreciated that in certain variations, the method 200 may include preparing the first solution and/or the third solution. The preparing of the first solution may include diluting the diluted buffer stock solution to achieve the targeted osmolality. The preparing of the third solution may include contacting the one or more compounds to a stock solution.

With renewed reference to FIG. 2, the preparing 210 of the vesicle may include applying a centrifugal force to the chamber such that the water-in-oil monolayer emulsion droplets can pass through the second and then the first solutions and the lipid bilayer that includes the block co-polymers around the water-in-oil monolayer emulsion droplet can be formed. In certain variations, the chamber may be a three-dimensional printed generally cylindrical or oval chamber, which is opened at the top and mounted onto a tabletop stirring motor that rotates at about 1,200 revolutions per minute (rpm). Although not illustrated, it should be appreciated that, in certain variations, the method 200 may also include one or more washing steps that removes any remaining third solution from around the formed vesicles.

In certain variations, the as-prepared vesicles may be mixed with a precursor hydrogel solution that is used to form the hydrogel. The precursor hydrogel solution may form the hydrogel upon the addition of one or more crosslinkers. The one or more crosslinkers may include, for example, calcium solution. The precursor hydrogel may include a macromer solution. In certain variations, the macromer solution may be a solution of polymers in aqueous media. The macromer solution may include about 60 wt. % per volume or more, optionally about 70 wt. % per volume or more, optionally about 80 wt. % per volume or more, and in certain aspects, optionally about 90 wt. % per volume or more, of water. The macromer solution may have an osmolality that is greater than or equal to about 1% to less than or equal to about 50%, and in certain aspects, optionally greater than or equal to about 1% to less than or equal to about 20%, greater than the third solution. For example, in certain variations, the macromer solution may have an osmolality greater than or equal to about 202 mOsm/kg to less than or equal to about 1,500 mOsm/kg, and in certain aspects, optionally greater than or equal to about 202 mOsm/kg to less than or equal to about 1,200 mOsm/kg.

Although not illustrated, it should appreciated that, in certain variations, the method 200 may include preparing the macromer solution by contacting the polymers to the aqueous media. The polymers may be a biocompatible polymer like those detailed above. For example, the macromer solution may include a polymer selected from the group consisting of: dextran, starch, cellulose, alginate, hyaluronic acid, chitosan, chitin, pectin, collagen, fibronectin, gelatin, polyethylene glycol (PEG), poly(vinyl alcohol) (PVA), poly(2-hydroxyethyl methacrylate) (PHEMA), polyvinylpyrrolidone (PVP), polyimide (PI), polyamide (PA), polyacrylic acid (PAA), polyurethane (PU), polyesters, derivatives thereof, and combinations thereof.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A material for therapeutics or tissue engineering, the material comprising:
a biocompatible hydrogel matrix; and
a plurality of vesicles distributed within the biocompatible hydrogel matrix, each of the vesicles defining a shell and a core region, wherein:
the shell comprises a bilayer comprising a lipid and a block co-polymer and is configured to release one or more compounds from the core region in response to an external stimulus; and
the core region comprises the one or more compounds to be released into the biocompatible hydrogel matrix.

2. The material of claim 1, wherein the biocompatible hydrogel matrix comprises:
(i) a polysaccharide selected from the group consisting of dextran, starch, cellulose, alginate, hyaluronic acid, chitosan, chitin, pectin, derivatives thereof, and combinations thereof;
(ii) a polypeptide selected from the group consisting of collagen, fibronectin, gelatin, derivatives thereof, and combinations thereof;
(iii) a synthetic polymer selected from the group consisting of: polyethylene glycol (PEG), poly(vinyl alcohol) (PVA), poly(2-hydroxyethyl methacrylate) (PHEMA), polyvinylpyrrolidone (PVP), polyimide (PI), polyamide (PA), polyacrylic acid (PAA), polyurethane (PU), polyesters, derivatives thereof, and combinations thereof; or
(iv) any combination of (i)-(iii).

3. The material of claim 1, wherein the biocompatible hydrogel matrix comprises less than 40 wt. % of a polymer selected from the group consisting of: dextran, starch, cellulose, alginate, hyaluronic acid, chitosan, chitin, pectin, collagen, fibronectin, gelatin, polyethylene glycol (PEG), poly(vinyl alcohol) (PVA), poly(2-hydroxyethyl methacrylate) (PHEMA), polyvinylpyrrolidone (PVP), polyimide (PI), polyamide (PA), polyacrylic acid (PAA), polyurethane (PU), polyesters, derivatives thereof, and combinations thereof.

4. The material of claim 1, wherein the one or more compounds comprise a bioactive agent selected from the group consisting of: a cell adhesion factor, a growth factor, a peptide, a cytokine, a hormone, a pharmaceutical agent, matrix metalloproteinases (MMPs), plasmid DNA (pDNA), messenger ribonucleic acid (mRNA), strong interfering RNA (siRNA), micro RNA (miRNA), biochemicals, oligonucleotides, and combinations thereof.

5. The material of claim 1, wherein the one or more compounds comprise an active ingredient selected from the group consisting of: a therapeutic active ingredient, a systemic active ingredient, a chemotherapy active ingredient, a localized active ingredient, an oral care active ingredient, a nutritional active ingredient, a personal care active ingredient, a cosmetic active ingredient, a diagnostic imaging indicator agent, and combinations thereof.

6. The material of claim 1, wherein the lipid is selected from the group consisting of: dipalmitoylphosphatidylcholine (DPPC), 1,2-dioleoyl-sn-glycero-3-phosphocholine (DOPC), distearoylphosphatidylcholine (DSPC), phosphatidylcholine (POPC), 1,2-distearoyl-sn-glyero-3-phosphoethanolamine (DSPE), 1,2-dilauroyl-sn-glycero-3-phosphocholine (DLPC), cholesterol, egg phosphatidylcholine (Egg PC), derivatives thereof, and combinations thereof.

7. The material of claim 1, wherein the block co-polymer is selected from the group consisting of: PBD-b-PEO, PIB-b-PEO, TRI-PEO-b-PIB, oligo(Asp)-b-PPO, mPEG-b-P(MMA-grad-DMAEMA), mPEG-b-PCL, PEG-b-PCL, PEG-b-PLA, PEG-b-PBD, PDMS-b-PEO, PMOXA-b-PDMS-b-PMOXA, PEO-b-PPO-b-PEO, PGM-b-PPO-b-PGM, PEO-b-PDMS-b-PEO, PEO-b-PCL-b-PEO, PDMS-g-(PEO)₂, PDMS-g-(PEO)₂₋₃, PEO-b-PCL, PEG-b-PDMS-b-PEG, PEG-g-PDMS-g-PEG, derivatives thereof, and combinations thereof.

8. The material of claim 1, wherein a mole fraction of the lipid to the block co-polymer in the shell is greater than or equal to about 1:99 to less than or equal to about 99:1.

9. The material of claim 1, wherein the external stimulus increases a porosity of the shell to release the one or more compounds from the core region.

10. The material of claim 1, wherein the external stimulus ruptures the shell to release the one or more compound from the core region.

11. The material of claim 1, wherein the external stimulus is selected from the group consisting of: applied energy fields, electrical fields, magnetic fields, pH, humidity, stress, light, temperature, sound, cell secreted factors, enzymes, biomolecules, and combinations thereof.

12. The material of claim 1, wherein the external stimulus comprises a stress that is applied at greater than or equal to about 1 Pa to less than or equal to about 1,000 MPa.

13. The material of claim 1, wherein the shell further comprises a permanent super magnetic material and the external stimulus comprises a magnetic field.

14. The material of claim 1, wherein an average diameter of the plurality of vesicles is greater than 1 micrometer to less than or equal to about 100 micrometers.

15. The material of claim 1, wherein the material further comprises:
one or more cells distributed in the biocompatible hydrogel matrix.

16. A method for preparing a material for tissue engineering, the method comprising:
preparing a plurality of vesicles, each vesicle comprising a shell and a core region, the shell comprising a bilayer that comprises a lipid and a block co-polymer, and the core region comprising one or more compounds, the preparing comprises:
adding a first solution and a second solution to a chamber to form an oil-water interface, the second solution comprising the lipid and the block co-polymer,
adding water-in-oil monolayer emulsion droplets comprising the second solution and a third solution to the chamber, the third solution comprising the one or more compounds, and the first solution having an osmolality that is greater than or equal to about 1% to less than or equal to about 50% greater than the third solution, and
applying a centrifugal force to the chamber such that lipid and the block co-polymer for the bilayer; and
disposing the plurality of vesicles in a biocompatible hydrogel matrix.

17. The method of claim 16, wherein the first solution has an osmolality greater than or equal to about 202 mOsm/kg to less than or equal to about 1,500 mOsm/kg, the second solution comprises greater than or equal to about 1 mol. % to less than or equal to about 99 mol. % of the lipid and greater than or equal to about 1 mol. % to less than or equal to about 99 mol. % of the block co-polymer, and the third solution has an osmolality greater than or equal to about 200 mOsm/kg to less than or equal to about 1,000 mOsm/kg.

18-19. (canceled)

20. The method of claim 16, wherein the water-in-oil monolayer emulsion droplets comprise greater than or equal to about 80 vol. % to less than or equal to about 99 vol. % of the second solution and greater than or equal to about 1 vol. % to less than or equal to about 20 vol. % of the third solution.

21. The method of claim 16, wherein the lipid is selected from the group consisting of: dipalmitoylphosphatidylcholine (DPPC), 1,2-dioleoyl-sn-glycero-3-phosphocholine (DOPC), distearoylphosphatidylcholine (DSPC), phosphatidylcholine (POPC), 1,2-distearoyl-sn-glyero-3-phosphoethanolamine (DSPE), 1,2-dilauroyl-sn-glycero-3-phosphocholine (DLPC), cholesterol, egg phosphatidylcholine (Egg PC), derivatives thereof, and combinations thereof, the block co-polymer is selected from the group consisting of: PBD-b-PEO, PIB-b-PEO, TRI-PEO-b-PIB, oligo(Asp)-b-PPO, mPEG-b-P(MMA-grad-DMAEMA), mPEG-b-PCL, PEG-b-PCL, PEG-b-PLA, PEG-b-PBD, PDMS-b-PEO, PMOXA-b-PDMS-b-PMOXA, PEO-b-PPO-b-PEO, PGM-b-PPO-b-PGM, PEO-b-PDMS-b-PEO, PEO-b-PCL-b-PEO, PDMS-g-(PEO)₂, PDMS-g-(PEO)₂₋₃, PEO-b-PCL, PEG-b-PDMS-b-PEG, PEG-g-PDMS-g-PEG, derivatives thereof, and combinations thereof, and the biocompatible hydrogel matrix comprises less than 40 wt. % of a polymer selected from the group consisting of: dextran, starch, cellulose, alginate, hyaluronic acid, chitosan, chitin, pectin, collagen, fibronectin, gelatin, polyethylene glycol (PEG), poly(vinyl alcohol) (PVA), poly(2-hydroxyethyl methacrylate) (PHEMA), polyvinylpyrrolidone (PVP), polyimide (PI), polyamide (PA), polyacrylic acid (PAA), polyurethane (PU), polyesters, derivatives thereof, and combinations thereof.

22. (canceled)

23. The method of claim 16, wherein the one or more compounds comprises a bioactive agent selected from the group consisting of: a cell adhesion factor, a growth factor, a peptide, a cytokine, a hormone, a pharmaceutical agent, matrix metalloproteinases (MMPs), plasmid DNA (pDNA), messenger ribonucleic acid (mRNA), strong interfering RNA (siRNA), micro RNA (miRNA), biochemicals, oligonucleotides, and combinations thereof or the one or more compounds comprises a therapeutic active ingredient, a systemic active ingredient, a chemotherapy active ingredient, a localized active ingredient, an oral care active ingredient, a nutritional active ingredient, a personal care active ingredient, a cosmetic active ingredient, a diagnostic imaging indicator agent, and combinations thereof.

24-25. (canceled)
